(19) United States
(12) Patent Application Publication
(10) Pub. No.: US 2005/0008568 A1
(43) Pub. Date: Jan. 13, 2005

(54) Technetium or rhenium complexes, radiopharmaceutical products comprising them

(76) Inventors: Franck Mevellec, Le Chesnay (FR); Roberto Pasqualini, Clamart (FR); Henri Patin, Rennes (FR); Alain Roucoux, Rennes (FR); Nicolas Noiret, Rennes (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/496,109

(22) PCT Filed: Nov. 5, 2002
(86) PCT No.: PCT/IB02/04681

(30) Foreign Application Priority Data
Nov. 20, 2001 (FR) 0114991

(51) Int. Cl. A61K 51/00; C07F 1/00; C07F 13/00

(52) U.S. Cl. 424/1.11; 534/14; 556/13

(57) ABSTRACT

The invention relates to a technetium or rhenium complex of formula (I):
[M (R¹CS₃)₂L] (I)
in which M is Tc or Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹. The dithiolate ligand is preferably a dithiocarbamate.

The invention also relates to a radiopharmaceutical product comprising a complex of formula (I) with M representing ⁹⁹Tc, ¹⁸⁶Re or ¹⁸⁸Re.

A subject-matter of the present invention is novel technetium or rhenium complexes which can be used in radiopharmaceutical products for diagnosis or therapy and which exhibit the advantage of being able to comprise a biological vector.

It relates more particularly to technetium or rhenium complexes in which the metal is in the III oxidation state in the form of an M³+ ion.

Radiopharmaceutical products comprising these complexes are advantageous, in particular when they comprise a biological vector which renders them suitable for the diagnosis or for the therapy of various pathologies.

### STATE OF THE PRIOR ART

Radiopharmaceuticals form a class of radioactive compounds generally composed of a γ- or β-emitting radioelement. These molecules may be used alone, when they exhibit an intrinsic activity for the biological target, or else can be combined with an active biological molecule, conferring the desired tropism on the combination. Thus, the key point of this discipline is based on the development of novel radiopharmaceuticals specific for an organ, for a physiological function or for a pathology. For this, the isotope must first of all emit γ photons or β particles detectable by existing scintigraphic equipment. Among the radioelements most widely used in scintigraphy, technetium-99m emerges as being the radioisotope of choice for nuclear medicine.

For some years, efforts have also been directed at the search for novel radiopharmaceuticals with a therapeutic purpose. The radiotracers used in therapy are radiolabelled molecules designed to deliver, in the most specific way possible, therapeutic doses of ionizing rays to sites in the body exhibiting physiological disorders (cancerous tumours). This technique, referred to as “metabolic radiotherapy”, comes under nuclear medicine. In this context, rhenium and in particular the ¹⁸⁶Re and ¹⁸⁸Re isotopes prove to be highly promising radioelements.

Radiopharmaceutical products based on technetium or on rhenium are already known.

Thus, nitridobis(dithiocarbamato)Tc-99m complexes, in which the technetium is in the V oxidation state, have been provided in FR-A-2 698 272 [1] as products for the in vitro labelling of blood cells and in particular of leukocytes for the purpose of the anatomical locating of inflammatory or infectious foci. These complexes correspond to the formula:
(Tc≡N)L¹L²
in which L¹ and L² represent two ligands of formula: 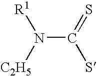
R¹ necessarily being an ethoxy group in at least one of the ligands L¹ and L², whereas it can be an ethyl group or an ethoxy group in the other of these ligands.

More recently, a rhenium complex with rhenium in the III oxidation state, and therefore a complex which is more stable chemically and thermodynamically than the preceding complexes, has been provided by F. Mevellec et al. in Inorg. Chem. Comm., 2, 1999, 230-233 [2], also for the in vitro labelling of leukocytes. This complex corresponds to the formula:
[Re(S₃CPh)₂(S₂CPh)]
and thus comprises three ligands, including two trithioperoxybenzoates and one dithiobenzoate.

The complexes disclosed in the document [1] and the document [2] exhibit a selective affinity with respect to leukocytes. Thus, placed in the presence of a blood sample, they are capable of selectively binding to the leukocytes present in this sample, this selectivity being expressed more particularly with respect to granulocytes, in the case of the complexes of the document [1], and lymphocytes, in the case of the complex of the document [2].

On the other hand, they do not comprise a biological molecule capable of acting as vector for them within a living organism and of directing them to a target other than an inflammatory or infectious focus.

Complexes based on rhenium sulphides have also been disclosed by Wei et al. in J. Am. Chem. Soc., 1990, 112, 6433-6434 [3], and by McConnachie and Stiefel in Inorg. Chem., 1997, 36, 6144-6145 [4] and Inorg. Chem., 1999, 38, 964-972 [5], but for use in the field of catalysis.

The Inventors therefore set themselves the target of providing technetium and rhenium complexes which, while exhibiting excellent chemical and thermodynamic stability, can comprise a biological vector capable of conferring on them a specificity with regard to specific cells, a specific physiological function or a specific pathology.

### DESCRIPTION OF THE INVENTION

A specific subject-matter of the present invention is novel technetium or rhenium complexes in which the technetium or rhenium is in the III oxidation state and which can additionally comprise a biological vector suitable for the diagnosis or therapeutic treatment to be carried out. According to the invention, these technetium or rhenium complexes correspond to the formula (I):
[M(R¹CS₃)₂L] (I)
in which M is Tc or Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹.

The complexes according to the invention thus exhibit the distinguishing feature of comprising both a radioelement in the III oxidation state, which confers on them a highly satisfactory chemical and thermodynamic stability, and a dithiolate ligand L which comprises a group R² different from the group R¹ of the other two ligands.

It is thus possible, in the case where it is desired to couple these complexes to a biological vector, to attach this vector to this dithiolate ligand alone and not to all the ligands and, consequently, to prevent steric hindrance which would have the effect of preventing the vector from binding to its target.

In accordance with the invention, the dithiolate ligand can be chosen from the dithiocarbamate, xanthate, dithiophosphate, dithiophosphonate, dithiophosphinate, dithiocarboxylate, 1,2-dithiolate and 1,2-dithiolene ligands.

The ligand L is preferably a dithiocarbamate of formula (II): 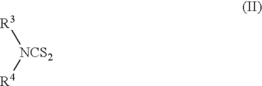
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

The presence of such a ligand is particularly advantageous as it can comprise either a group capable of reacting with a biological molecule, such as a hydroxyl, thiol, carboxylic acid, ester, amine, amide, thiocyanate or isothiocyanate group, or a group derived from a biological molecule which is optionally connected to the alkyl, aryl or aralkyl group of the ligand via a spacer.

This spacer can correspond to one of the following formulae:
(CH₂)ₙ, (CH₂O)ₙ, (CH₂S)ₙ and (CH₂NR)ₙ
in which R is as defined above and n is an integer ranging from 1 to 5.

The biological molecules capable of being attached to this ligand can be highly varied in nature. They can be, for example, antibodies, proteins, peptides, members of a ligands/receptors group, hormones or nucleic acids. Mention may be made, by way of examples, of molecules derived from somatostatin, such as octreotide, labels, ligands of the serotonin receptors, such as 1-(2-methoxyphenyl)piperazine, biotin, and the like.

In the case where the complexes comprise 1-(2-methoxyphenyl)piperazine, which has a high affinity for some cerebral receptors, the radiopharmaceutical products comprising such a complex will attach preferentially to the receptors specific for this biological molecule. The concentration of receptors of this type in the various cerebral regions can thus be measured experimentally. These radiopharmaceutical products can also be used for monitoring the inhibition of these receptors by other unlabelled molecules, for example medicaments or drugs, by measuring the variation in the concentration of receptors of this type due to the unlabelled molecule.

Depending on the biological vector used, the complexes according to the invention can be used as radiopharmaceuticals for detecting or treating cancers, neurodegenerative diseases (Parkinson's disease, Alzheimer's disease or multiple sclerosis) or dysfunctions of the cardiovascular system.

In accordance with the invention, the group R¹ of the sulphur-comprising ligands R¹CS₃ and the ligand L are chosen so that the latter is a more electronegative molecule than the said sulphur-comprising ligands, this being because the Inventors have found that this arrangement results in particularly stable complexes being obtained.

The group R¹ of the sulphur-comprising ligands R¹CS₃ can be an aliphatic, alkyl, cycloalkyl, aralkyl or aryl group. This group can be unsubstituted or substituted by one or more substituents chosen from halogen atoms, for example fluorine, the hydroxyl group, alkyl groups and alkoxy groups.

The alkyl groups used for R¹ can be linear or branched C₁ to C₁₂ groups, preferably groups having 3 to 13 carbon atoms.

The cycloalkyl groups used for R¹ preferably have 3 to 7 carbon atoms, for example 6 carbon atoms.

The aryl groups used for R¹ can be of the phenyl or naphthyl type.

The aralkyl groups used for R¹ can be of the C₆H₅(CH₂)ₙ type with n ranging from 1 to 3; preferably, n is equal to 1 or 2.

Preferably, according to the invention, the group R¹ is an optionally substituted aryl, aralkyl or cyclohexyl group.

Advantageously, when R¹ is an aryl group, it is chosen from the phenyl group, the phenyl group substituted by one or more methyl, ethyl, propyl, butyl, ethoxy, methoxy and/or hydroxyl groups and/or by one or more fluorine, chlorine, bromine and/or iodine atoms, the naphthyl group and the naphthyl group substituted by a group chosen from alkyl or alkoxy groups and halogen atoms.

In the case where R¹ is an aralkyl group, the latter is advantageously the benzyl or phenethyl group.

In the ligand L of dithiocarbamate type, R³ and R⁴ are chosen according to the use envisaged for the complex produced.

Mention may be made, as example of dithiocarbamate ligands used in the invention, of the ligands in which R³ and R⁴ have the meanings given below:
1) R³ and R⁴ are identical and represent a methyl, ethyl or ethoxy group;
2) R³ is the ethyl group and R⁴ is the hydroxyethyl group;
3) R³ and R⁴ form, with the nitrogen atom to which they are bonded, a piperidine, pyrrolidine, pyridine, piperazine, ethylpiperazine or morpholine ring;
4) R³ is a hydrogen atom and R⁴ represents the group of formula (III): 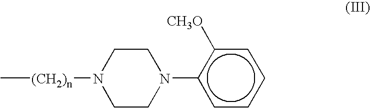
   in which n is an integer ranging from 1 to 6, preferably equal to 2; and
5) R³ and R⁴ form, together with the nitrogen atom to which they are bonded, the group of formula (X): 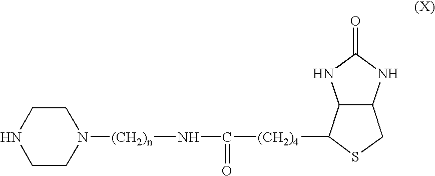
   in which n is an integer ranging from 1 to 6, preferably equal to 2.

The technetium and rhenium complexes described above can be used in radiopharmaceutical products.

Consequently, another subject-matter of the invention is a radiopharmaceutical product comprising a technetium or rhenium complex as described above in which M is ^{99m}Tc, ¹⁸⁶Re or ¹⁸⁸Re.

Another subject-matter of the invention is a process for the preparation of the technetium or rhenium complexes corresponding to formula (I).

According to a first embodiment of this process, the technetium or rhenium complex of formula (I) is obtained by carrying out the following stages:
reacting a salt of formula (IVa) or (IVb):
   (MO₄)⁻Zₐ⁺ (IVa)
   [MOCl₄]⁻Zₐ⁺ (IVb)
   in which M is as defined above and Zₐ⁺ is a pharmaceutically acceptable cation, with a reducing agent, and
adding, to the reaction mixture, a dithiocarboxylate of formula (V):
   (R¹CS₂)⁻Z_{b}⁺ (V)
   in which R¹ is as defined above and Z_{b}⁺ represents a pharmaceutically acceptable cation, and a salt L⁻X⁺ where L is as defined above and X⁺ is a cation chosen from sodium and potassium.

According to a second embodiment of the process of the invention, the complexes of formula (I) are obtained from a technetium or rhenium complex of formula (VI):
[M(R¹CS₃)₂(R¹CS₂)] (VI)
in which M and R¹ are as defined above, by bringing this complex into contact with a salt L⁻X⁺, where L is as defined above and X⁺ is a cation chosen from sodium and potassium, in order to exchange the ligand R¹CS₂ with the ligand L.

The complexes of formula (VI) used as starting materials in this second embodiment of the process of the invention can be prepared by a process comprising the following stages:
reacting a salt of formula (IVa):
   (MO₄)⁻Zₐ⁺ (IVa)
   in which M is as defined above and Zₐ⁺ is a pharmaceutically acceptable cation, with a reducing agent, and
adding, to the reaction mixture, a dithiocarboxylate of formula (V):
   (R¹CS₂)⁻Z_{b}⁺ (V)
   in which R¹ is as defined above and Z_{b}⁺ represents a pharmaceutically acceptable cation.

In these various methods of preparation, the pharmaceutically acceptable cations used for Zₐ⁺ can be alkali metal or alkaline earth metal ions, for example sodium, ammonium ions and quaternary ammonium ions, such as NH₄ and NBu₄, with Bu representing the butyl group.

The pharmaceutically acceptable cations used for Z_{b}⁺ can be chosen from MgX⁺, where X is a halogen atom, such as Br or Cl, quaternary ammonium cations and alkali metal ions, such as sodium.

The quaternary ammonium cations can be, for example, of the NR₄ type, where R is an alkyl group, for example methyl. Use may also be made of quaternary ammonium cations of the piperidinium type of formula C₅H₁₀NH₂⁺.

In both embodiments of the process of the invention, the reducing agent used can be of various types. Use may in particular be made of a reducing agent composed of a tin salt in combination with a complexing agent having a higher complexing power for the tin than that of the dithiocarboxylate.

This complexing agent can be of the phosphonate, polyphosphate and polyaminocarboxylic acid type. Mention may be made, as examples of such complexing agents, of ammonium or alkali metal or alkaline earth metal pyrophosphates, ammonium or alkali metal or alkaline earth metal glucoheptonates, ammonium or alkali metal diethylenetriaminepentaacetates, ammonium or alkali metal or alkaline earth metal ethylenediaminetetraacetates, ammonium or alkali metal or alkaline earth metal 1,2-diaminopropane-N,N,N′,N′-tetraacetates, ammonium or alkali metal or alkaline earth metal gluconates, ammonium or alkali metal or alkaline earth metal methylenediphosphonates, ammonium or alkali metal or alkaline earth metal hydroxymethylenediphosphonates, and ammonium or alkali metal or alkaline earth metal citrates.

Use may be made in the invention, by way of example, of a tin salt composed of tin chloride in combination with a complexing agent chosen from calcium gluconate, 1,2-diaminopropane-N,N,N′,N′-tetraacetic acid and a dithiocarbazate DTCZ.

Use may also be made, according to the invention, of reducing agents composed of a phosphine or of one of its derivatives in combination with hydrochloric acid.

Mention may be made, as example of phosphine and of phosphine derivative, of triphenylphosphine and sodium triphenylphosphine-tri-meta-sulphonate P(C₆H₄SO₃)₃Na₃.

In the process of the invention, the metal M, which was initially in the VII oxidation state, is reduced to the III oxidation state, while a portion of the dithiocarboxylate ligand is oxidized to trithioperoxycarboxylate.

The amounts of reducing agent used in this process are chosen according to the amount of pertechnetate or perrhenate initially introduced.

In the case of the pertechnetate Tc^{99m}, for activities of 30 MBq to 4 GBq, use may be made of amounts of reducing agent ranging from 0.01 to 1 mg in the case of SnCl₂.2H₂O, in the presence of an excess of complexing agent with respect to the tin chloride.

When a triphenylphosphine is used as reducing agent, the amounts used are of the order of 0.1 to 5 mg, in the case of pure triphenylphosphine, and of 0.2 to 10 mg, in the case of sodium triphenylphosphine-tri-meta-sulphonate. An aqueous HCl solution is added with these reducing agents in order to obtain 1×10⁻² to 1×10⁻¹ mol/l of HCl in the reaction medium.

Despite the similarity in the chemical properties between the pertechnetate and the perrhenate, it is known that, for the reduction reaction, the latter ion requires larger amounts of reducing agent than those employed for the pertechnetate ion.

Furthermore, in the case where the radioactive metal is rhenium-186, an isotope having a low specific activity, the amount of perrhenate used is greater in order to obtain the same activity; consequently, to reduce this species, larger amounts of reducing agent will be used than in the case of the rhenium-188 isotope.

Thus, use may be made of 0.1 to 5 mg of reducing agent in the case of SnCl₂.2H₂O, 0.1 to 10 mg in the case of pure triphenylphosphine and 0.2 to 20 mg in the case of sodium triphenylphosphine-tri-meta-sulphonate.

A sufficient amount of dithiocarboxylate and optionally of L⁻X⁺ salt, preferably dissolved in physiological saline, is subsequently added to the reaction medium. The reaction of the ligand(s) with the pertechnetate or perrhenate is carried out under hot conditions, for example at a temperature of 100° C.

In the first embodiment of the process of the invention, the operation is carried out in an organic solvent, such as dichloromethane, or in water by adding, to the solution of the salts of the ligand L and of R¹CS₂, a solution of the salt of formula (MO₄)⁻Zₐ⁺ in the same solvent.

In the second embodiment of the process of the invention, an exchange reaction is carried out between the technetium or rhenium complex of formula (VI) and a salt of the ligand L. For this exchange reaction, a salt of the ligand L in solution in an organic solvent, such as methanol, or in water is added to the complex of formula (VI) in solution in an organic solvent, such as dichloromethane, or in suspension in water.

According to the invention, when the ligand L comprises a group capable of reacting with a biological molecule, the process can comprise an additional stage consisting in reacting the complex formed above with a biological molecule in order to attach it to the ligand L via this group. The biological molecule can also be introduced onto the ligand L beforehand, in order to directly obtain a complex comprising this biological molecule.

A further subject-matter of the invention is a kit for the preparation of a radiopharmaceutical product comprising a complex of formula (I):
[M(R¹CS₃)₂L] (I)
in which M is ^{99m}Tc, ¹⁸⁶Re or ¹⁸⁸Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹, characterized in that it comprises:
a first bottle comprising a tin salt in combination with a complexing agent, or a phosphine and hydrochloric acid,
a second bottle comprising a dithiocarboxylate of formula (R¹CS₂)⁻Z_{b}⁺ in which R¹ is as defined above and Z_{b}⁺ represents a pharmaceutically acceptable cation, and
a third bottle comprising a salt L⁻X⁺ where L is as defined above and X⁺ is a cation chosen from sodium and potassium.

When the kit is intended to implement the first embodiment of the process of the invention, the first and the second bottles can be replaced by a single bottle and, in this case, the kit comprises:
a first bottle comprising a tin salt in combination with a complexing agent, or a phosphine and hydrochloric acid, and
a second bottle comprising a dithiocarboxylate of formula (R¹CS₂)⁻Z_{b}⁺ in which R¹ is as defined above and Z_{b}⁺ represents a pharmaceutically acceptable cation, and a salt L⁻X⁺ where L is as defined above and X⁺ is a cation chosen from sodium and potassium.

Generally, in both embodiments of the kit, the first bottle comprises tin chloride SnCl₂.2H₂O in combination with a complexing agent chosen from calcium gluconate, 1,2-diaminopropane-N,N,N′,N′-tetraacetic acid and a dithiocarbazate DTCZ. According to an alternative implementation, this first bottle comprises triphenylphosphine or sodium triphenylphosphine-tri-meta-sulphonate, and hydrochloric acid.

In both embodiments of the kit, the latter can additionally comprise a bottle comprising a biological molecule.

The radiopharmaceutical products comprising the complexes of the invention are particularly advantageous as they can be adapted to various pathologies, depending on the nature of the ligand L and of the biological molecule with which it is combined.

Thus, it is possible to obtain, in accordance with the invention, radiopharmaceutical products labelled with a suitable biological vector.

Furthermore, such radiopharmaceutical products with technetium ^{99m}Tc or with rhenium ¹⁸⁶Re or ¹⁸⁸Re can be prepared in less than one hour from a kit comprising three bottles respectively comprising the reducing agent (tin salt-gluconate), the dithiocarboxylate (R¹CS₂)⁻Z_{b}⁺ and the salt of the ligand L, for example a dithiocarbamate.

Other characteristics and advantages of the invention will become more clearly apparent on reading the following examples, given, of course, by way of illustration and without implied limitation.

### DETAILED DESCRIPTION OF THE EMBODIMENTS

The following Examples 1 to 11 illustrate the preparation of rhenium and technetium complexes in accordance with the invention.

Examples 1 to 6 illustrate the first embodiment of the process of the invention. Examples 7 to 11 illustrate the second embodiment of the process of the invention.

Examples 12 to 14 illustrate the preparation of dithiocarbamate ligands of use in the preparation of the complexes of the invention.

### EXAMPLE 1

### Preparation of bis(trithioperoxybenzoato)-(diethyldithiocarbamato)rhenium (III) [Re(PhCS₃)₂(Et₂NCS₂)]

A mixture of 0.407 g (1.7 mmol) of piperidinium phenyldithiocarboxylate salt and 0.383 g (1.7 mmol) of sodium diethyldithiocarbamate in the minimum amount of dichloromethane (20 ml) is added dropwise to a solution comprising 0.100 g (0.170 mmol) of [ReOCl₄][NBu₄] in dichloromethane (5 ml). After stirring for 6 hours at ambient temperature, the solvent is evaporated and the residue, taken up in dichloromethane, is chromatographed on a column of silica gel (eluent: petroleum ether/dichloromethane: 70/30) and then recrystallized from a petroleum ether/dichloromethane mixture.

0.090 g of complex is thus obtained in the form of khaki green crystals (yield of 75%).

The characteristics of this complex are as follows:
M=705.11;
melting point M.p.=164-166° C.;
R_{f}=0.50 (petroleum ether/CH₂Cl₂: 70/30);

¹H NMR (CDCl₃): 1.19 (t, J=7.1 Hz, 6H, CH₃), 3.60 (m, 4H, CH₂), 7.31 (t, J=8.1 Hz, 2H, H₈), 7.53 (t, J=8.1 Hz, 4H, H₇), 8.07 (d, J=8.6 Hz, 4H, H₆).

¹³C NMR (CDCl₃): 12.8 (CH₃), 44.4 (CH₂), 128.1 and 132.4 (C₆ and C₇), 132.8 (C₈), 134.9 (C₅), 204.5 (NCS₂), 233.4 (CS₃).

Mass spectrometry (FAB): m/z=704.7: [M⁺.]; 551.8: [M^{+•}-(PhCS₂)]. Elemental analysis: empirical formula: C₁₉H₂₀NS₈Re Experimental: % C=32.44 % H=2.87 % S=36.41 Calculated: % C=32.35 % H=2.86 % S=36.36

Infrared (KBr disc): 2 963 (w), 2 921 (w), 2 852 (w), 1 504 (s), 1 482 (m), 1 454 (m), 1 440 (s), 1 377 (w), 1 354 (w), 1 300 (w), 1 275 (m), 1 241 (m), 1 208 (m), 1 179 (w), 1 149 (m), 1 078 (w), 1 028 (w), 1 001 (s), 990 (s, ν_{C═S}) 908 (m), 755 (s), 687 (s), 544 (s, ν_{S—S}) 454 (m), 399 (m, ν_{Re—S}).

### EXAMPLE 2

### Preparation of bis(trithioperoxybenzoato)-(dimethyldithiocarbamato)rhenium(III) [Re(PhCS₃)₂(Me₂NCS₂)]

The same procedure as in Example 1 is followed in preparing this complex, using sodium dimethyldithiocarbamate instead of sodium diethyldithiocarbamate. 0.080 g of green crystals of the complex (yield=69% ) is thus obtained. The characteristics of the complex are as follows:
M=677.05;
M.p.=160-162° C.;
R_{f}=0.45 (petroleum ether/CH₂Cl₂: 70/30);

¹H NMR (CDCl₃): 3.24 (s, 6H, CH₃), 7.31 (t, J=7.6 Hz, 2H, Hₐᵣ), 7.52 (t, J=8.1 Hz, 4H, Hₐᵣ), 8.07 (dd, J=8.1 Hz and J=2.5 Hz, 4H, Hₐᵣ).

¹³C NMR (CDCl₃): 39.5 (CH₃), 128.1 and 132.4 (CH_{o and m}), 132.9 (CHₚ), 134.9 (C_{q}), 207.1 (NCS₂), 233.8 (CS₃). Elemental analysis: empirical formula: C₁₇H₁₆NS₈Re Experimental: % C=30.21 % H=2.40 % S=37.85 Calculated: % C=30.16 % H=2.38 % S=37.89

Infrared (KBr disc): 1 594 (w), 1 569 (w), 1 533 (w), 1 481 (m), 1 441 (m), 1 393 (s), 1 329 (w), 1 261 (m), 1 252 (m), 1 148 (m), 1 098 (w), 1 027 (w), 1 009 (m), 990 (s, ν_{C═S}) 775 (s), 681 (s), 544 (s, ν_{S—S}) 454 (m), 399 (m, ν_{Re—S}).

### EXAMPLE 3

### Preparation of bis(trithioperoxybenzoato)-(N-piperidyldithiocarbamato)rhenium(III) [Re(PhCS₃)₂(C₅H₁₀NCS₂)]

The same procedure as in Example 1 is followed in preparing this rhenium complex, using piperidinium piperidyldithiocarbamate instead of sodium diethyldithiocarbamate.

The piperidyldithiocarbamate is obtained in the following way.

4.25 g (0.050 mol, 5 ml) of piperidine are dissolved in 100 ml of ether in a two-necked flask and 2.40 g (0.060 mol) of NaOH are added to the solution with stirring. The mixture obtained is cooled to −15° C. using a bath of liquid nitrogen and 3.95 g (0.052 mol, 3.2 ml) of carbon disulphide are added dropwise to the solution. The mixture is maintained at −15° C. for 30 minutes. After returning to ambient temperature with stirring, the solid obtained is filtered off, washed with ether and dried under vacuum, and then recrystallized from an isopropanol/ether (1:2) mixture.

5.03 g of white crystals, comprising 41% of piperidinium piperidyldithiocarbamate and a very small amount of sodium piperidyldithiocarbamate, are thus obtained.

The characteristics of the piperidinium salt are as follows:

¹H NMR: 1.55 (m, 8H, 2H₃, 4H₅ and 2H₆), 1.69 (m, 4H, 4H₂), 3.07 (t, 4H, H₄, J₄₋₅=5.6 Hz), 4.17 (t, 4H, H₁, J₁₋₂=5.3 Hz).

¹³C NMR: 21.1 (C₆), 21.9 (C₃), 23.3 (C₅), 25.2 (C₂), 44.2 (C₄), 52.6 (C₁), 202.2 (C—S).

Infrared spectrum (ν in cm⁻¹): 2 930 (s, CH₂), 2 852 (m, CH₂), 1 449 (w, CH₂), 1 409 (s, C—N), 1 218 (s, C═S).

By following the procedure of Example 1 and by using this dithiocarbamate, 0.085 g of complex in the form of green crystals (yield=70% ) is obtained.

The characteristics of the complex are as follows:
M=716.96;
M.p.=196-198° C.;
R_{f}=0.46 (petroleum ether/CH₂Cl₂: 70/30);

¹H NMR (CDCl₃): 1.60 (s, br, 4H, CH₂), 1.65 (s, br, 2H, CH₂), 3.74 (s, br, 4H, NCH₂), 7.31 (t, J=7.6 Hz, 2H, Hₐᵣ), 7.54 (t, J=7.6 Hz, 4H, Hₐᵣ), 8.07 (d, J=8.1 Hz, 4H, Hₐᵣ).

¹³C NMR (CDCl₃): 23.8 (CH₂), 25.3 (CH₂), 47.6 (CH₂), 128.1 and 132.4 (CHₐᵣ), 132.9 (CHₐᵣ), 134.9 (Cₐᵣ), 203.5 (NCS₂), 233.5 (CS₃).

Mass spectrometry (FAB): m/z=716.891914: [M⁺.]; 563.9: [M⁺.-(PhCS₂)]; 531.9: [Re—(PhCS₃)]. Elemental analysis: empirical formula: C₂₀H₂₀NS₈Re Experimental: % C=33.29 % H=2.79 % S=35.81 Calculated: % C=33.50 % H=2.81 % S=35.77

Infrared (KBr disc): 2 963 (w), 2 921 (w), 2 852 (w), 1 498 (s), 1 441 (s), 1 261 (s), 1 243 (m), 1 096 (s), 1 023 (s), 800 (s, νC═S), 755 (m), 678 (m), 544 (s), ν_{S—S}) 454 (m), 399 (m, ν_{Re—S}).

### EXAMPLE 4

### Preparation of bis(trithioperoxybenzoato)-(N-morpholinodithiocarbamato)rhenium(III) [Re(PhCS₃)₂(O(CH₂CH₂)₂NCS₂)]

The same procedure as in Example 1 is followed, using sodium morpholinodithiocarbamate instead of sodium diethyldithiocarbamate.

0.069 g of complex is thus obtained in the form of green crystals (yield=55% ).

The characteristics of the complex are as follows:
M=719.04;
M.p.>260°0 C.;
R_{f}=0.16 (petroleum ether/CH₂Cl₂: 70/30);

¹H NMR (CDCl₃): 3.63 (t, J=4.1 Hz, 4H, NCH₂), 3.77 (t, J=4.0 Hz, 4H, OCH₂), 7.48 (m, 6H, Hₐᵣ), 8.02 (dd, J=1.0 and 8.4 Hz, 4H, Hₐᵣ).

¹³C NMR (CDCl₃): 46.1 (NCH₂), 65.9 (OCH₂), 128.1, 132.5 and 133.0 (CHₐᵣ), 134.9 (Cₐᵣ), 205.8 (NCS₂), 248.6 (CS₃). Elemental analysis: empirical formula: C₁₉H₁₈NOReS₈. Experimental: % C=32.02 % H=2.55 % S=35.81 Calculated: % C=31.70 % H=2.50 % S=35.70

Infrared (KBr disc): 1 492 (s), 1 442 (m), 1 428 (m), 1 298 (w), 1 268 (w), 1 235 (s), 1 183 (w), 1 114 (w), 1 028 (w), 1 008 (s, ν_{C—S}), 994 (m, ν_{C—S}), 908 (w), 882 (w), 757 (m), 680 (w), 551 (w), 544 (s, ν_{S—S}), 454 (m), 399 (m, ν_{Re—S}).

### EXAMPLE 5

### Preparation of bis(trithioperoxybenzoato)-(N-ethyl-N-(2-hydroxyethyl)dithiocarbamato)rhenium(III) [Re(PhCS₃)₂( (HOCH₂CH₂)N(Et)CS₂)]

The same procedure as in Example 1 is followed, except that sodium N-ethyl-N-(2-hydroxyethyl)dithiocarbamate is used instead of sodium diethyldithiocarbamate.

0.063 g of complex is thus obtained in the form of green crystals (yield=50% ).

The characteristics of this complex are as follows:
M=721.06;
M.p.=196-198° C.;
R_{f}=0.46 (petroleum ether/CH₂Cl₂: 70/30);

¹H NMR (CDCl₃): 0.81 (t, J=7.1 Hz, 3H, CH₃), 1.60 (s, br, 2H, NCH₂CH₃), 1.65 (s, br, 2H, CH₂CH₂N), 3.67 (q, J=7.0 Hz, 2H, OCH₂CH₂N), 7.26 (t, J=7.6 Hz, 2H, Hₐᵣ), 7.47 (t, J=7.6 Hz, 4H, Hₐᵣ), 8.01 (d, J=8.1 Hz, 4H, Hₐᵣ).

¹³C NMR (CDCl₃): 23.8 (CH₃), 24.6 (NCH₂), 25.3 (NCH₂), 47.6 (OCH₂), 128.1 and 132.4 (CHₐᵣ), 132.9 (CHₐᵣ), 134.9 (Cₐᵣ), 203.5 (NCS₂), 233.5 (CS₃). Elemental analysis: empirical formula: C₁₉H₂₀NORes₈. Experimental: % C=31.51 % H=2.79 % S=35.77 Calculated: % C=31.60 % H=2.80 % S=35.60

Infrared (KBr disc): 2 963 (w), 2 921 (w), 2 852 (w), 1 499 (s), 1 482 (m), 1 442 (s), 1 261 (w), 1 245 (s), 1 180 (w), 1 118 (w), 1 029 (w), 1 004 (s, ν_{C═S}) 992 (s, νf_{C—S}) 757 (s), 580 (m), 544 (s, ν_{S—S}) 454 (m), 399 (m, ν_{Re—S).}

### EXAMPLE 6

### Preparation of bis(trithioperoxy-naphthoato)(diethyldithiocarbamato)rhenium(III) [Re(C₁₀H₇CS₃)₂(Et₂NCS₂)]

The same procedure as in Example 1 is followed, except that piperidinium dithionaphthoate is used instead of the piperidinium phenyldithiocarboxylate salt.

0.70 g of the complex is thus obtained in the form of green crystals (yield=50% ).

The characteristics of this complex are as follows:
M=805.18;
M.p.=218° C.;
R_{f}=0.53 (petroleum ether/CH₂Cl₂: 70/30);

¹H NMR (CDCl₃): 1.20 (t, J=7.1 Hz, 6H, CH₃), 3.80 (q, J=7.1 Hz, 4H, NCH₂), 7.85 (dd, J=1.5 Hz and J=6.9 Hz, 2H, Hₐᵣ), 7.90 (t, J=6.6 Hz and J=7.9 Hz, 2H, Hₐᵣ), 7.98 (t, J=7.4 Hz and J=7.9 Hz, 2H, Hₐᵣ), 8.07 (d, J=6.9 Hz, 2H, Hₐᵣ), 8.20 (d, J=8.1 Hz, 2H, Hₐᵣ), 8.25 (d, J=7.6 Hz, 2H, Hₐᵣ), 8.44 (d, J=8.1 Hz, 2H, Hₐᵣ).

¹³C NMR (CDCl₃): 12.8 (CH₃), 44.6 (CH₂), 125.8, 126.4, 127.3, 128.5, 130.4, 130.5, 131.2, 132.9, 133.5 and 133.5 (10 Cₐᵣ), 207.1 (CS₂), 233.6 (CS₃). Elemental analysis: empirical formula: C₂₇H₂₄NReS₈. Experimental: % C=40.44 % H=3.02 % S=31.96 Calculated: % C=40.30 % H=3.00 % S=31.90

Infrared (KBr disc): 1 505 (s), 1 456 (w), 1 436 (m), 1 390 (w), 1 352 (w), 1 276 (m), 1 240 (m), 1 208 (w), 1 152 (w), 1 076 (w), 1 002 (s, ν_{C═S}), 856 (w), 796 (s), 770 (s), 453 (w), 428 (w), 544 (s, ν_{S—S}) 454 (m), 399 (m, ν_{Re—S).}

### EXAMPLE 7

### Preparation of bis(trithioperoxybenzoato)-(diethyldithiocarbamato)rhenium(III) [Re(PhCS₃)₂(Et₂NCS₂)]

In this example, the complex of Example 1 is prepared by following the second embodiment of the process of the invention.

### a) Preparation of the Complex [Re(S₃CPh)₂(S₂CPh)]

0.300 g (1.25 mmol) of piperidinium phenyldithiocarboxylate salt, dissolved in the minimum amount of dichloromethane (30 ml), is added dropwise to solution comprising 0.560 g (0.208 mmol) of [ReO₄][NH₄], 0.118 g (0.622 mmol) of SnCl₂.2H₂O and 5.0 g (27.1 mmol) of citric acid in dichloromethane (10 ml). After stirring for 2 hours at ambient temperature, the solvent is evaporated and the residue, taken up in dichloromethane, is chromatographed on a column of silica gel (eluent: petroleum ether/dichloromethane: 60/40) and then recrystallized from a petroleum ether/dichloromethane mixture.

0.090 g of complex is thus obtained in the form of green crystals (yield=61% ).

The characteristics of this complex are as follows:
M=709.90;
M.p.=205-207° C.;
R_{f}=0.50 (petroleum ether/CH₂Cl₂: 80/20);

¹H NMR (CDCl₃): 7.29 (m, J=3.5 Hz, 5H, H₄, H₅ and H₁₀), 7.44 (t, J=8.0 Hz, 4H, H₉), 7.63 (dd, J=8.0 Hz and J=2.5 Hz, 2H, H₃), 7.96 (dd, J=8.5 Hz and J=1.5 Hz, 4H, H₈).

¹³C NMR (CDCl₃): 123.7 (C₃), 126.4 (C₄), 127.2 (C₈), 130.8 (C₉), 132.1 (C₅), 132.3 (C₁₀), 133.9 (C₇), 141.7 (C₂), 232.8 (CS_{2 6}), 237.6 (CS_{2 1}).

Mass spectrometry (FAB): m/z=709.8527: [M⁺.]; 556.9: [M-(PhCS₂)]⁺; 493.0: [Re(PhCS₂)₂]⁺. Elemental analysis: empirical formula: C₂₁H₁₅S₈Re. Experimental: % C=35.34 % H=2.13 % S=35.99 Calculated: % C=35.35 % H 2.13 % S=36.03

Infrared (KBr disc): 1 482 (m), 1 442 (s), 1 332 (m), 1 311 (w), 1 263 (s), 1 234 (w), 1 179 (w), 1 156 (w), 1 096 (m), 1 028 (m), 997 (s, ν_{C—S}), 947 (w), 908 (w), 802 (m), 755 (s), 681 (w), 652 (w), 544 (s, ν_{S—S}) 454 (m), 399 (m, ν_{Re—S}).

### b) Preparation of the Final Complex

0.032 g (1.41 mmol) of sodium diethyldithiocarbamate, in the minimum amount of methanol (10 ml), is added dropwise to a solution of 0.020 g (0.028 mmol) of [Re(S₃CPh)₂(S₂CPh)] in dichloromethane (10 ml). After stirring for 1 hour at ambient temperature, the solvent is evaporated and the residue, taken up in dichloromethane, is chromatographed on a column of silica gel (eluent: petroleum ether/dichloromethane: 70/30) and then recrystallized from a petroleum ether/dichloromethane mixture.

0.020 g of complex is thus obtained in the form of khaki green crystals, which corresponds to a quantitative yield.

The characteristics of the complex are identical to those in Example 1.

### EXAMPLE 8

### Preparation of bis(trithioperoxybenzoato)-(dimethyldithiocarbamato)rhenium(III) [Re (PhCS₃)₂(Me₂NCS₂)]

In this example, the same procedure as in Example 7 is followed, using sodium dimethyldithiocarbamate instead of sodium diethyldithiocarbamate.

0.015 g of complex is thus obtained in the form of green crystals, which corresponds to a yield of 79%.

The characteristics of the complex are identical to those in Example 2.

### EXAMPLE 9

### Preparation of bis(trithioperoxybenzoato)-(N-piperidyldithiocarbamato)rhenium(III) [Re(PhCS₃)₂(C₅H₁₀NCS₂)]

In this example, the same procedure as in Example 7 is followed for preparing the complex of Example 3, using piperidyldithiocarbamate instead of diethyldithio-carbamate.

0.015 g of complex is thus obtained in the form of green crystals, which corresponds to a yield of 75%.

The characteristics of the complex are identical to those in Example 3.

### EXAMPLE 10

### Preparation of bis(trithioperoxybenzoato)-(diethyldithiocarbamato)technetium(III) [Tc(PhCS₃)₂(Et₂NCS₂)]

In this example, the second embodiment of the process of the invention is followed for preparing the technetium complex.

### a) Preparation of the Complex [Tc(PhCS₃)₂(PhCS₂)]

0.4 to 0.8 GBq of sodium pertechnetate NaTcO₄ is added to a flask comprising 75.0 mg of calcium gluconate, 0.75 mg of SnCl₂.2H₂O and 25.0 mg of sodium chloride dissolved in 10 ml of physiological saline. The mixture is stirred at ambient temperature for 10 minutes, then 20 mg of sodium dithiobenzoate NaPhCS₂, dissolved in 1.0 ml of physiological saline under warm conditions, are added and the solution is heated at 100° C. for an additional 15 minutes.

The complex obtained has the following characteristics:
R_{f}=0.62 (petroleum ether/CH₂Cl₂: 70/30).

### b) Preparation of the Final Complex

0.098 g (0.410 mmol) of sodium diethyldithiocarbamate in the minimum amount of methanol (10 ml) is added dropwise to a solution of 0.051 g (0.082 mmol) of [Tc(S₃CPh)₂(S₂CPh)] in dichloromethane (10 ml). After stirring for 1 hour at ambient temperature, the precipitate is filtered off, washed several times with methanol, chromatographed on a column of silica gel (eluent: petroleum ether/dichloromethane: 70/30) and then recrystallized from a petroleum ether/dichloromethane mixture.

0.033 g of complex is thus obtained in the form of pink crystals, which corresponds to a yield of 66%.

The characteristics of the complex are as follows:
M=616.85;
R_{f}=0.50 (petroleum ether/CH₂Cl₂: 70/30);

¹H NMR (CDCl₃): 1.17 (t, 6H, CH₃), 3.66 (m, 4H, CH₂), 7.46 (m, 6H, H₈+H₆), 8.10 (m, 4H, H₇).

¹³C NMR (CDCl₃): 12.6 (CH₃), 44.5 (CH₂), 128.6 and 129.5 (C₆ and C₇), 132.2 (C₈), 138.2 (C₅), 200.0 (NCS₂), 225.7 (CS₃).

Infrared (KBr disc): 2 952 (m), 2 915 (m), 2 854 (m), 1 504 (s), 1 499 (m), 1 458 (m), 1 440 (m), 1 376 (w), 1 274 (m), 1 207 (m), 1 180 (w), 1 148 (m), 1 074 (w), 1 001 (m), 999 (s, ν_{C═S)} 904 (s), 756 (s), 731 (m), 683 (s).

### EXAMPLE 11

### Preparation of bis(trithioperoxybenzoato)-([4-(2-methoxyphenyl)piperazin-1-ylethyl]dithio-carbamato)technetium(III)

In this example, the same procedure as in Example 10 is followed for preparing this technetium complex, using, instead of sodium diethyldithiocarbamate, the dithiocarbamate of formula (VII):

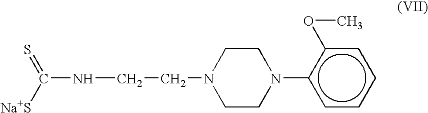

A biological molecule, 1-(2-methoxyphenyl)piperazine, which has a high affinity for some receptors located on the neurotransmitters, is thus introduced into the complex.

The dithiocarbamate of formula (VII) is prepared in the following way.

The starting material is 2-bromoethylamine and the final salt is obtained in four stages:
protection of the primary amine;
introduction of the 1-(2-methoxyphenyl)piperazine;
deprotection of the amine; and
formation of the dithiocarbamate functional group.

### a) Protection of 2-bromoethylammonium bromide

This reaction corresponds to the following scheme:

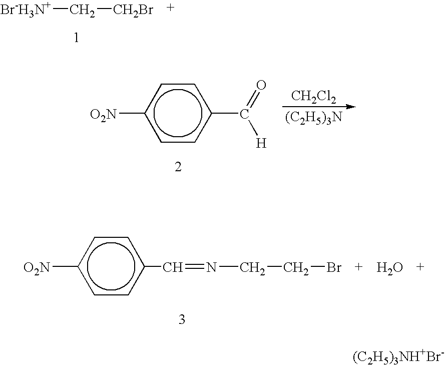

14 mmol (2.87 g) of 2-bromoethylammonium bromide 1 and 14 mmol (1.95 ml, 1.42 g) of triethylamine are dissolved with stirring in 100 ml of dichloromethane in a 150 ml two-necked flask. 10 mmol (1.51 g) of para-nitrobenzaldehyde 2 are added to the solution and the mixture is kept stirred overnight.

The solvent is evaporated and the resulting yellow solid is taken up in 50 ml of dichloromethane and 50 ml of water. The organic phase is extracted with 3×25 ml of dichloromethane and washed with 3×25 ml of water, then dried over magnesium sulphate and concentrated under vacuum. 2.16 g of yellow solid 3 are obtained. The yield is 80%.

The characteristics of the product 3 are as follows:

¹H NMR (CDCl₃) δ in ppm: 3.74 (t, 2H, H₇, J₆₋₇=6.0 Hz), 4.10 (td, 2H, H₆, J₅₋₆=1.2 Hz), 7.94 (m, 2H, H₃), 8.29 (m, 2H, H₂, J₂₋₃=8.8 Hz), 8.39 (m, 1H, H₅, J₅₋₆=1.2 Hz).

¹³C NMR (CDCl₃) δ in ppm: 32.0 (C₇), 62.1 (C₆), 123.5 and 128.9 (C₂ and C₃), 140.8 and 148.9 (C₁ and C₄), 160.6 (C₅).

Infrared spectrum (ν in cm⁻¹): 3 100-3 075 (w, aromatic CH), 2 882-2 827 (w, aliphatic CH), 1 645 (m, C═N), 1 603 (m, C═C), 1 523 (s, NO₂), 1 424 (w, CH₂), 1 342 (s, NO₂), 1 266 (m, C—N), 567 (w, C—Br).

### b) Introduction of the 1-(2-methoxyphenyl)piperazine

This reaction corresponds to the following scheme: 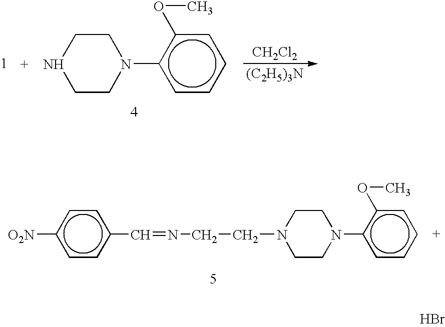
12.02 mmol (2.31 g) of 1-(2-methoxyphenyl)piperazine 4 and 12.02 mmol (1.21 g, 1.68 ml) of triethylamine are dissolved in 100 ml of dichloromethane in a 100 ml round-bottomed flask. 8.02 mmol (2.06 g) of the Schiff base 3 are added to the solution with stirring. The mixture is kept stirred at ambient temperature overnight.

The solvent is evaporated and the yellow residue obtained is taken up in 50 ml of dichloromethane and 50 ml of water. The organic phase is extracted with 3×20 ml of dichloromethane and washed with 3×20 ml of water, then dried over magnesium sulphate and concentrated under vacuum. The residue obtained is dissolved in 30 ml of ether and the resulting pale yellow solid 5 (1.79 g) is filtered off and washed with ether. The yield is 60%.

The characteristics of the product 5 are as follows:

¹H NMR (CDCl₃) δ in ppm: 2.81 (m, 6H, H₇ and H₈), 3.12 (m, 4H, H₉), 3.88 (s, 3H, H₁₆), 3.89 (m, 2H, H₆), 6.94 (m, 4H, aromatic H), 7.91 (m, 2H, H₃, J₂₋₃=8.8 Hz), 8.28 (m, 2H, H₂), 8.41 (m, 1H, H₅).

¹³C NMR (CDCl₃) δ in ppm: 50.2 and 53.4 (C₈ and C₉), 55.0 (C₁₆), 58.3 and 59.0 (C₆ and C₇), 110.8, 117.8, 120.6 and 122.6 (C₁₁ to C₁₄), 123.5 and 128.4 (C₂ and C₃), 140.9 and 148.7 (C₁ and C₄), 141.3 and 151.9 (C₁₀ and C₁₅), 159.2 (C₅).

Infrared spectrum (ν in cm⁻¹): compound 5: 2 944 (m, CH₃), 2 811 (m, CH₂), 1 648 (m, C═N), 1 601 (m, C═C), 1 522 (s, NO₂), 1 458 (m, CH₂, CH₃), 1 342 (s, NO₂), 1 239 (m, C—O).

### c) Deprotection of the Amine According to the Following Reaction Scheme

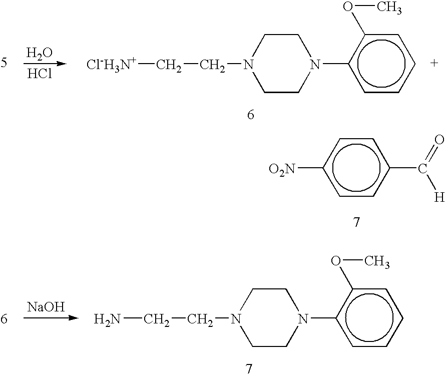

4.34 mmol (1.60 g) of the product 5 are dissolved in 30 ml of dichloromethane in a 100 ml round-bottomed flask. 13 ml of a molar hydrochloric acid solution are added with stirring to the solution. Stirring is then maintained for 2 hours at ambient temperature. The aqueous phase is extracted with 3×10 ml of water and neutralized with 13 ml of a molar sodium hydroxide solution. The organic phase is taken up in 3×10 ml of dichloromethane, then washed with 3×5 ml of water, dried over magnesium sulphate and concentrated under vacuum. After evaporating the solvent, 0.53 g of a yellow oil 7 are obtained, which corresponds to a yield of 51%.

The characteristics of the product 7 are as follows:

¹H NMR (CDCl₃) δ in ppm: 2.51 (t, 2H, H₂, J₁₋₂=6.1 Hz), 2.67 (m, 4H, H₃), 2.85 (t, 2H, H₁, J₁₋₂=6.1 Hz), 3.10 (m, 4H, H₄), 3.86 (m, 3H, H₁₁), 6.93 (m, 4H, aromatic H).

¹³C NMR (CDCl₃) δ in ppm: 38.2 (C₁), 50.3 and 53.1 (C₃ and C₄), 55.0 (C₁₁), 60.6 (C₂), 110.8, 117.8, 120.6 and 122.5 (C₆ to C₉), 141.0 and 152.0 (C₅ and C₁₀).

### d) Formation of the Dithiocarbamate Functional Group According to the Following Reaction Scheme

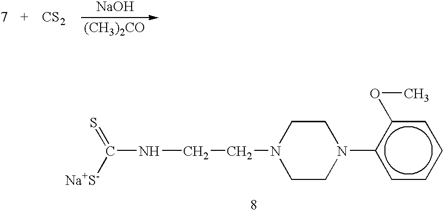

2.25 mmol (0.53 g) of the amine 7 are dissolved in 2 ml of water in a 100 ml two-necked flask. 10 ml of an aqueous sodium hydroxide solution (2.60 mmol) are added to the solution, followed by 12 ml of acetone, and the resulting mixture is stirred until a clear solution is obtained. 2.80 mmol (0.21 g, 0.17 ml) of carbon disulphide 8 are added dropwise and with stirring to the mixture. The solution is cooled using an ice bath and maintained at low temperature (with an ice bath) for 5 hours and with stirring, and then the mixture is placed at 0° C. overnight. The solvent is subsequently evaporated and the residue obtained is washed with ether. 0.48 g of product 8 is obtained, i.e. a yield of 64%.

The characteristics of the thiocarbamate 8 are as follows:

¹H NMR (CDCl₃) δ in ppm: 2.63 (m, 6H, H₂ and H₃), 2.98 (s, 4H, H₄), 3.67 (t, 2H, H₁, J₁₋₂=6.8 Hz), 3.76 (s, 3H, H₁₁), 7.01 (m, 4H, aromatic H).

¹³C NMR (CDCl₃) δ in ppm: 50.2 and 52.8 (C₃ and C₄), 55.0 (C₁₁), 56.2 (C₂), 110.8, 117.8, 120.6 and 122.8 (C₆ to C₉), 151.9 (C₅-C₁₀).

Infrared spectrum (ν in cm⁻¹): 3 392 (NH), 2 939 (w, CH₃), 2 829 (w, CH₂), 1 498 (s, NH), 1 458 (m, C═C), 1 377 (w, CH₂, CH₃), 1 234 (s, C—O), 1 115 (m, C═S).

Sodium 2-[4-(2-methoxyphenyl)piperazin-1-yl]ethyl-dithiocarbamate is obtained with a good yield.

### EXAMPLE 12

### Preparation of a dithiocarbamate Comprising Biotin

Biotin is a vitamin present at a low concentration in the blood which can be used to diagnose or treat certain tumours (in particular, tumours of the abdomen). The method used can consist in injecting, into the body, an antibody to which has been attached a molecule specific for a substrate, in this instance avidin, which has a high affinity for biotin. These antibodies become located at the tumour. The biotin-comprising technetium complex is then injected into the body and will become located preferentially on the antibodies introduced above, which makes possible visualization of the tumour.

The dithiocarbamate used for the preparation of the complex corresponds to the following formula:

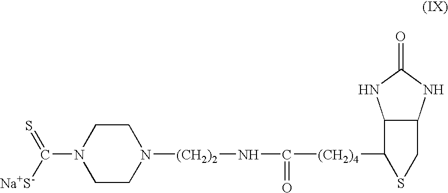

The starting material is the primary amine 1-(2-aminoethyl)piperazine and stages analogous to those described in Example 11 are carried out.

### a) Protection of the Primary Amine

14 mmol (1.81 g, 1.84 ml) of 1-(2-aminoethyl)piperazine are dissolved in 100 ml of dichloromethane with stirring in a 150 ml two-necked flask. 10 mmol (1.51 g) of para-nitrobenzaldehyde are added to the solution and the mixture is kept stirred overnight at ambient temperature. The solvent is evaporated and the orange solid obtained is taken up in 50 ml of dichloromethane and 50 ml of water. The organic phase is extracted with 3×25 ml of dichloromethane and then washed with 3×25 ml of water, dried over magnesium sulphate and concentrated under vacuum. An orange solid is recovered (2.00 g, i.e. a yield of 76% ).

The characteristics of this product are as follows:

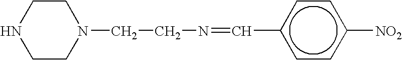

¹H NMR (CDCl₃) δ in ppm: 2.55 (m, 4H, H₂), 2.72 (m, 2H, H₃, J₃₋₄=7.0 Hz), 2.91 (m, 4H, H₁, J₁₋₂=4.8 Hz), 3.84 (m, 2H, H₄), 7.89 (m, 2H; H₇), 8.27 (m, 2H, H₈), 8.38 (m, 1H, H₅, J₄₋₅=1.3 Hz).

¹³C NMR (CDCl₃) δ in ppm: 45.7 and 54.4 (C₁ and C₂), 59.1 (C₄), 123.5 and 128.4 (C₇ and C₈), 141.3 and 148.6 (C₆ and C₉), 159.2 (C₅).

### b) Formation of the Sodium Dithiocarbamate Salt

7.63 mmol (2.0 g) of the product obtained in a) are dissolved in 100 ml of dichloromethane in a 150 ml two-necked flask. 12 mmol (0.48 g) of sodium hydroxide are added to the solution with stirring. The mixture is cooled to −15° C. using a bath of liquid nitrogen and 11 mmol (0.84 g, 0.66 ml) of carbon disulphide are added dropwise to the solution with stirring. After returning to ambient temperature, the mixture is stirred overnight. The precipitate obtained is washed with 50 ml of dichloromethane and then 50 ml of ether and filtered off. It is dissolved in 100 ml of a dichloromethane/water (1:1) mixture and the aqueous phase is extracted and concentrated under vacuum. A brown solid is obtained.

### c) Deprotection of the Amine

The solid obtained at the end of stage b) is dissolved in 50 ml of dichloromethane and 20 ml of water. 8 mmol (0.32 g) of sodium hydroxide are added to the solution and stirring is maintained for one hour. The aqueous phase is extracted with 20 ml of water and concentrated under vacuum. A brown solid is obtained (1.57 g, yield=69% ).

It corresponds to the formula:

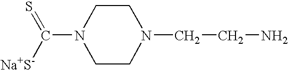

¹H NMR (D₂O) δ in ppm: 2.41 (m, 2H, H₃), 2.49 (t, 4H, H₂, J₁₋₂=5.2 Hz), 2.69 (m, 2H, H₄), 4.26 (t, 4H, H₁, J₁₋₂=5.1 Hz).

¹³C NMR (D₂O) δ in ppm: 36.9 (C₄), 49.9 and 51.6 (C₁ and C₂), 58.8 (C₃), 208.1 (C═S).

### d) Coupling of the Biotin According to the Following Reaction Scheme

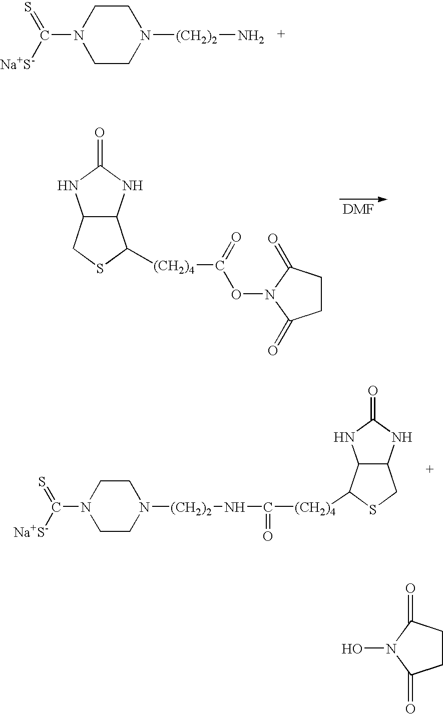

The biotin is first of all esterified with N-hydroxysuccinimide and then reacted with the thiocarbamate obtained in stage c) in dimethylformamide (DMF). The biotin-comprising dithiocarbamate thus obtained is insoluble in this solvent and can be recovered.

### EXAMPLE 13

### Preparation of the pyrrolidyldithio-carbamate

In this example, the same procedure as in Example 3 is followed for preparing this dithiocarbamate, using:
0.025 mol (1.78 g, 2 ml) of pyrrolidine instead of the piperidine;
0.030 mol of NaOH (1.20 g); and
0.026 mol (1.98 g, 1.6 ml) of CS₂.

1.94 g of a mixture comprising 37% of sodium pyrrolidyldithiocarbamate and 63% of pyrrolidinium pyrrolidyldithiocarbamate are thus obtained, which mixture has the following characteristics:

¹H NMR: 1.90 (m, 6.8H, H₂ and H₄), 3.18 (m, 2.6H, H₃, J₃₋₄=7.3 Hz), 3.68 (m, 4.2H, H₁, J₁₋₂=7.0 Hz).

¹³C NMR: 23.3 (C₄), 25.2 (C₂), 45.2 (C₃), 54.6 (C₁), 202.2 (C═S).

Infrared spectrum (ν in cm⁻¹): 2 943 (m, CH₂), 2 868 (m, CH₂), 1 420 (s, C—N), 1 400 (m, CH₂), 1 160 (m, C═S).

### EXAMPLE 14

### Preparation of sodium 1-ethylpiperazinyl-dithiocarbamate

In this example, the same procedure as in Example 3 is followed for preparing this dithiocarbamate, using:
0.025 mol (2.85 g, 3 ml) of 1-ethylpiperazine instead of the piperidine;
0.030 mol (1.20 g) of NaOH; and
0.026 mol (1.98 g, 1.6 ml) of CS₂.

3.11 g of sodium 1-ethylpiperazinyldithiocarbamate are thus obtained, which product exhibits the following characteristics:

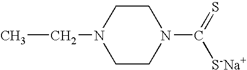

¹H NMR: 1.01 (t, 3H, H₄), 2.44 (q, 2H, H₃, J₃₋₄=7.3 Hz), 2.52 (m, 4H, H₂), 4.28 (m, 4H, H₁).

¹³C NMR: 10.0 (C₄), 50.8 (C₃), 49.8 and 59.9 (C₁ and C₂), 202.1 (C═S).

Infrared spectrum (ν in cm⁻¹): 2 969 (w, CH₃), 2 818 (m, CH₂), 1 415 (s, C—N), 1 227 (s, C═S).

### References Cited

[1] FR-A-2 698 272
[2] F. Mevellec et al., Inorg. Chem. Comm., 2, 1999, 230-233
[3] Wei et al., J. Am. Chem. Soc., 1990, 112, 6433-6434
[4] McConnachie and Stiefel, Inorg. Chem., 1997, 36, 6144-6145
[5] McConnachie and Stiefel, Inorg. Chem., 1999, 38, 964-972

1-44. (Cancel)

45. Technetium or rhenium complex of formula (I):
[M(R¹CS₃)₂L] (I)
in which M is Tc or Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹.

46. Complex according to claim 45, in which L is chosen from dithiocarbamate, xanthate, dithiophosphate, dithiophosphonate, dithiophosphinate, dithiocarboxylate, 1,2-dithiolate and 1,2-dithiolene ligands.

47. Complex according to claim 46, in which the ligand L is a dithiocarbamate of formula (II): 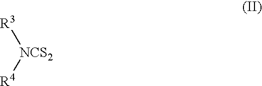
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

48. Complex according to claim 45, in which the ligand L is more electronegative than the groups R¹CS₃.

49. Complex according to claim 45, in which R¹ is the phenyl group.

50. Complex according to claim 45, in which R¹ is a linear or branched C₁ to C₁₂ alkyl group.

51. Complex according to claim 45, in which R¹ represents the naphthyl group or the naphthyl group substituted by a group chosen from alkyl groups, alkoxy groups and halogen atoms.

52. Complex according to claim 45, in which R¹ is chosen from phenyl groups substituted by one or more methyl, ethyl, propyl, butyl, methoxy, ethoxy and/or hydroxyl groups and/or by one or more fluorine, chlorine, bromine and/or iodine atoms.

53. Complex according to claim 45, in which R¹ is the cyclohexyl, benzyl or phenethyl group.

54. Complex according to claim 47, in which R³ and R⁴ are a methyl, ethyl or ethoxy group.

55. Complex according to claim 47, in which R³ is the ethyl group and R⁴ is the hydroxyethyl group.

56. Complex according to claim 47, in which R³ and R⁴ form, with the nitrogen atom to which they are bonded, a piperidine, pyrrolidine, pyridine, ethylpiperazine or morpholine ring.

57. Complex according to claim 47, in which R³ is a hydrogen atom and R⁴ represents the group of formula (III): 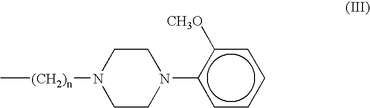
in which n is an integer ranging from 1 to 6, preferably equal to 2.

58. Complex according to claim 47, in which R³ and R⁴ form, with the nitrogen atom to which they are bonded, the group of formula (X): 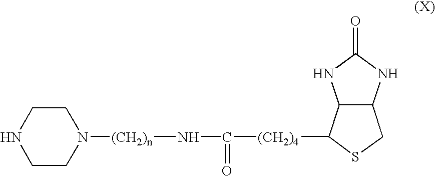
in which n is an integer ranging from 1 to 6, preferably equal to 2.

59. Radiopharmaceutical product comprising a complex according to claim 45, in which M is ^{99m}Tc, ¹⁸⁶Re or ¹⁸⁸Re.

60. Process for the preparation of a technetium or rhenium complex of formula (I):
[M(R¹CS₃)₂L] (I)
in which M is Tc or Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹,
which comprises the following stages:
reacting a salt of formula (IVa) or (IVb):
(MO₄)⁻Zₐ⁺ (IVa)
[MOCl₄]⁻Zₐ⁺ (IVb)
in which M is as defined above and Zₐ⁺ is a pharmaceutically acceptable cation, with a reducing agent, and
adding, to the reaction mixture, a dithiocarboxylate of formula (V):
(R¹CS₂)⁻Z_{b}⁺ (V)
in which R¹ is as defined above and Z_{b}⁺ represents a pharmaceutically acceptable cation, and a salt L⁻X⁺ where L is as defined above and X⁺ is a cation chosen from sodium and potassium.

61. Process for the preparation of a technetium or rhenium complex of formula (I):
[M(R¹CS₃)₂L] (I)
in which M is Tc or Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹,
which comprises bringing a technetium or rhenium complex of formula (VI):
[M(R¹CS₃)₂(R¹CS₂)] (VI)
in which M and R¹ are as defined above, into contact with a salt L⁻X⁺, where L is as defined above and X⁺ is a cation chosen from sodium and potassium, in order to exchange the ligand R¹CS₂ with the ligand L.

62. Process according to claim 60, in which the ligand L comprises a group capable of reacting with a biological molecule and in that the process comprises an additional stage consisting in reacting the complex formed with a biological molecule in order to attach it to the ligand L.

63. Process according to claim 61, in which the complex of formula (VI):
[M(R¹CS₃)₂(R¹CS₂)] (VI)
is prepared by carrying out the following stages:
reacting a salt of formula (IVa):
(MO₄)⁻Zₐ⁺ (IVa)
in which M is as defined above and Zₐ⁺ is a pharmaceutically acceptable cation, with a reducing agent, and
adding, to the reaction mixture, a dithiocarboxylate of formula (V):
(R¹CS₂)⁻Z_{b}⁺ (V)
in which R¹ is as defined above and Z_{b}⁺ represents a pharmaceutically acceptable cation.

64. Process according to claim 60, in which Z_{b}⁺ represents a cation chosen from MgX⁺, where X is a halogen atom, an alkali metal ion, a quaternary ammonium cation or the piperidinium cation.

65. Process according to claim 60, in which the reducing agent is a tin salt in combination with a complexing agent, or an HCl/phosphine mixture.

66. Process according to claim 65, in which the tin salt is tin chloride and the complexing agent is calcium gluconate, 1,2-diaminopropane-N,N,N′,N′-tetraacetic acid and a dithiocarbazate DTCZ.

67. Process according to claim 65, in which the phosphine is triphenylphosphine or sodium triphenylphosphine-tri-meta-sulphonate.

68. Process according to claim 60, in which the salt L⁻X⁺ corresponds to the formula (XI): 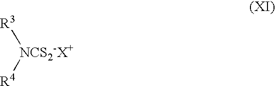
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

69. Kit for the preparation of a radiopharmaceutical product comprising a complex of formula (I):
[M(R¹CS₃)₂L] (I)
in which M is ^{99m}Tc, 186Re or 188Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹,
which comprises:
a first bottle comprising a tin salt in combination with a complexing agent, or a phosphine and hydrochloric acid,
a second bottle comprising a dithiocarboxylate of formula (R¹CS₂)⁻Z_{b}⁺ in which R¹ is as defined above and Z_{b}⁺ represents a pharmaceutically acceptable cation, and
a third bottle comprising a salt ^{L}⁻X⁺ where L is as defined above and X⁺ is a cation chosen from sodium and potassium.

70. Kit for the preparation of a radiopharmaceutical product comprising a complex of formula (I):
[M(R¹CS₃)₂L] (I)
in which M is ^{99m}Tc, ¹⁸⁶Re or ¹⁸⁸Re, R¹ represents an alkyl, cycloalkyl, aralkyl or aryl group which is unsubstituted or substituted by one or more substituents chosen from halogen atoms, the hydroxyl group, alkyl groups and alkoxy groups, and L is a dithiolate ligand, with the exception of the ligand of formula R²CS₂ in which R² is identical to R¹,
which comprises:
a first bottle comprising a tin salt in combination with a complexing agent, or a phosphine and hydrochloric acid, and
a second bottle comprising a dithiocarboxylate of formula (R¹CS₂)⁻Z_{b}⁺ in which R¹ is as defined above and Z_{b}⁺ represents a pharmaceutically acceptable cation, and a salt L⁻X⁺ where L is as defined above and X⁺ is a cation chosen from sodium and potassium.

71. Kit according to claim 69, in which the first bottle comprises tin chloride SnCl₂.2H₂O in combination with a complexing agent chosen from calcium gluconate, 1,2-diaminopropane-N,N,N′,N′-tetraacetic acid and a dithiocarbazate DTCZ.

72. Kit according to claim 69, in which the first bottle comprises triphenylphosphine or sodium triphenylphosphine-tri-meta-sulphonate, and hydrochloric acid.

73. Kit according to claim 69, which additionally comprises a bottle comprising a biological molecule.

74. Kit according to claim 69, in which the salt L⁻X⁺ is a dithiocarbamate of formula (XI): 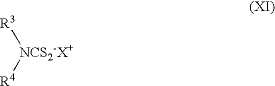
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

75. Kit according to claim 74, in which R³ is a hydrogen atom and R⁴ represents the group of formula (III): 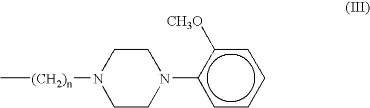
in which n is an integer ranging from 1 to 6, preferably equal to 2.

76. Kit according to claim 74, in which R³ and R⁴ form, with the nitrogen atom to which they are bonded, the group of formula (X): 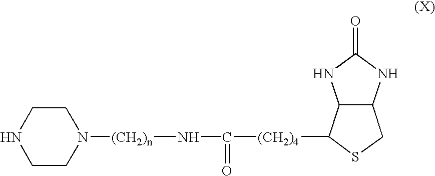
in which n is an integer ranging from 1 to 6, preferably equal to 2.

77. Process according to claim 61, in which the ligand L comprises a group capable of reacting with a biological molecule and in that the process comprises an additional stage consisting in reacting the complex formed with a biological molecule in order to attach it to the ligand L.

78. Process according to claim 63, in which Z_{b}⁺ represents a cation chosen from MgX⁺, where X is a halogen atom, an alkali metal ion, a quaternary ammonium cation or the piperidinium cation.

79. Process according to claim 63, in which the reducing agent is a tin salt in combination with a complexing agent, or an HCl/phosphine mixture.

80. Process according to claim 79, in which the tin salt is tin chloride and the complexing agent is calcium gluconate, 1,2-diaminopropane-N,N,N′,N′-tetraacetic acid and a dithiocarbazate DTCZ.

81. Process according to claim 79, in which the phosphine is triphenylphosphine or sodium triphenylphosphine-tri-meta-sulphonate.

82. Process according to claim 61, in which the salt L⁻X⁺ corresponds to the formula (XI): 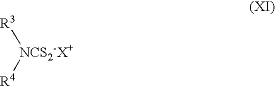
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

83. Kit according to claim 70, in which the first bottle comprises tin chloride SnCl₂.2H₂O in combination with a complexing agent chosen from calcium gluconate, 1,2-diaminopropane-N,N,N′,N′-tetraacetic acid and a dithiocarbazate DTCZ.

84. Kit according to claim 70, in which the first bottle comprises triphenylphosphine or sodium triphenylphosphine-tri-meta-sulphonate, and hydro-chloric acid.

85. Kit according to claim 70, which additionally comprises a bottle comprising a biological molecule.

86. Kit according to claim 70, in which the salt L⁻X⁺ is a dithiocarbamate of formula (XI): 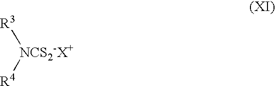
in which R³ and R⁴, which can be identical or different, represent a hydrogen atom, a C₁ to C₁₀, preferably C₁ to C₅, alkyl group, a C₆ to C₁₀ aryl group or a C₇ to C₁₂ aralkyl group, the alkyl, aryl or aralkyl groups optionally comprising one or more groups chosen from OH, SH, COOH, COOR, NH₂, NHR, NR₂, CONH₂, CONHR, CONR₂, NCSR and SCNR where the R groups, which can be identical or different, represent an alkyl or aryl group, groups capable of reacting with a biological molecule and groups derived from a biological molecule which are optionally connected to the alkyl, aryl or aralkyl group via a spacer, or in which R³ and R⁴ form, together with the nitrogen atom to which they are bonded, a heterocycle having from 3 to 5 carbon atoms optionally comprising another heteroatom chosen from O, S and N, this heterocycle being unsubstituted or substituted by one or more C₁ to C₁₀ alkyl, C₆ to C₁₀ aryl or C₇ to C₁₂ aralkyl groups, the alkyl, aryl or aralkyl groups optionally comprising one or more groups chosen from OH, SH, COOH, COOR, NH₂, NHR, NR₂, CONH₂, CONHR, CONR₂, NSCR and SCNR where the R groups, which can be identical or different, represent an alkyl or aryl group, groups capable of reacting with a biological molecule and groups derived from a biological molecule which are optionally connected to the alkyl, aryl or aralkyl group via a spacer.

87. Kit according to claim 86, in which R³ is a hydrogen atom and R⁴ represents the group of formula (III): 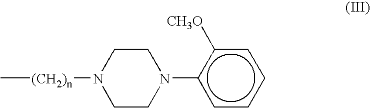
in which n is an integer ranging from 1 to 6, preferably equal to 2.

88. Kit according to claim 86, in which R³ and R⁴ form, with the nitrogen atom to which they are bonded, the group of formula (X): 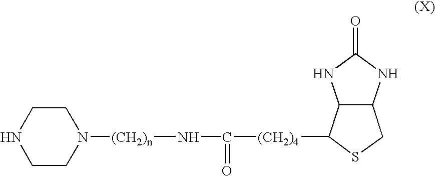
in which n is an integer ranging from 1 to 6, preferably equal to 2.
